(19) United States
(12) Patent Application Publication
(10) Pub. No.: US 2025/0223243 A1
(43) Pub. Date: Jul. 10, 2025

(54) SYNTHESIZING AN ORGANIC HIGH EXPLOSIVE IN A FLOW REACTOR

(71) Applicant: BAE SYSTEMS plc, London (GB)

(72) Inventors: Andy Oden Burn, USK Monmouthshire (GB); Matthew Paul Didsbury, USK Monmouthshire (GB); Ian Ewart Paterson Murray, Aylesbury Buckinghamshire (GB); Niall John McWhir, USK Monmouthshire (GB); Daniel Jubb, Aylesbury Buckinghamshire (GB); Christopher Jones, Aylesbury Buckinghamshire (GB); Nicola Kennedy, Aylesbury Buckinghamshire (GB); Stuart Kennedy, Aylesbury Buckinghamshire (GB)

(73) Assignee: BAE SYSTEMS plc, London (GB)

(21) Appl. No.: 18/687,632

(22) PCT Filed: Aug. 30, 2022
(86) PCT No.: PCT/GB2022/052217

§ 371 Date: Feb. 28, 2024

(30) Foreign Application Priority Data
Sep. 2, 2021 (GB) 2112498.7

(51) Int. Cl. C06B 49/00; B01J 19/00; C06B 21/00; C07D 251/06; C07D 257/02

(52) U.S. Cl. CPC C06B 49/00; B01J 19/0093; C06B 21/0008; C07D 251/06; C07D 257/02; B01J 2219/00862; B01J 2219/00869

(57) ABSTRACT

A method of synthesising an organic high explosive includes the steps of
i) providing a first solution A,
ii) providing a second solution B,
wherein the admixture of solution A and solution B are selected such that they are capable upon formation of the admixture of reacting together to provide an organic high explosive, and
iii) causing the solution A and B to be mixed and passed through a flow reactor to create an admixture,
wherein the flow reactor includes a pipe, wherein the internal diameter of the pipe is selected such that it is less than the critical diameter of the organic high explosive, thereby preventing detonation of the formed organic high explosive in said flow reactor.

The following invention relates to methods of producing explosives from the direct nitration explosive precursors by flow synthesis. Particularly to a method of producing RDX and HMX.

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims.

According to a first aspect of the invention there is provided

A method of synthesising an organic high explosive, comprising the steps of
i) providing a solution A comprising a nitrating agent,
ii) providing a solution B comprising an explosive precursor reagent,
wherein the admixture of solution A and solution B are selected such that they are capable upon formation of the admixture of reacting together to provide an organic high explosive,
iii) determining the critical diameter of the organic high explosive,
iv) wherein the flow reactor comprises a pipe, selecting the internal diameter of the pipe such that it is less than the critical diameter of the organic high explosive, thereby preventing detonation of the formed organic high explosive in said flow reactor,
v) causing the solution A and B to be mixed and passed through a flow reactor to create the admixture

The solution A comprises a nitrating agent, such as for example nitric acid, nitrites and combinations thereof.

The solution B comprises an explosive precursor reagent, explosive precursors are well known in the art, and are often controlled access reagents, as they are readily nitrated with high percentage nitric acid, such as fuming or 99% conc nitric acid.

The explosive precursor may be an aromatic compound, phenylamines, cycloamine, toluene, Octahydro-1,3,5,7-tetranitro-1,3,5,7-tetrazocine (TAT), 1,3,5-triacetyl-1,3,5-triazacyclohexanes (TRAT), 1,5-Dinitroendomethylene-1,3,5,7-tetraazacyclooctane (DPT), triazole, and hexamethylenetetramine. The formation of the TAT, TRAT and DPT may require several synthetic steps, but can be safely made using conventional batch techniques as they are not energetic materials.

The solution A and/or B or a solution C which may be mixed at stage ii) further comprises catalysts, strong acids, de-hydrating agents, and acid anhydrides. Strong acids other than nitric acid may be sulphuric acid. De-hydrating agents may be P₂O₅. Acid anhydrides may be acetic acid anhydride or trifluoroacetic anhydride.

The nitration reaction is typically exothermic and the flow reactor may be temperature controlled to ensure that the explosive material formed does not run to detonation.

The admixture transitions through the flow reactor, to which a solution D may be added to work up the reactant to provide a precipitate of said explosive material or a salt thereof. The solution D may quench the acid. Solution D, may comprise cooled water, to cause precipitation.

The batch synthesis of explosives is very well regulated due to the explosive hazard. However, the batch pathway for industrial synthesis causes the formation of hundreds of kilos of material to be formed in a reactor. This will cause the building to have a very large safety radius. The use of flow synthesis allows low kilogram production, such that the explosive material may be collected remote from the stored solutions A and/or B and/or C, such as to reduce the hazard of an event. Preferably the location of the collected remote is behind a blast wall or explosive magazine. The use of continuous production of smaller mass of explosive material prevents the build-up of a several hundred kilo batch in one place.

The critical diameter of explosives may be readily characterised, using well defined tests. Publically available sources show that the critical diameter is generally of the order of greater than 1 mm, depending on the test used.

The critical diameters of the pure explosive are often modified, by the use of binders to reduce their sensitivity (IM explosives), and the critical diameter of such a composition such as PBX (polymer bonded explosives) may be different to the pure explosive material.

The use of flow synthesis allows the selection of the internal diameter of the pipe used in the flow synthesis, the internal diameter of the flow reactor pipe must be less than the critical diameter of the explosive material being synthesised to mitigate against sustained detonation in the flow reactor. The critical diameter may be determined, and the selection of the diameter of the flow reactor pipe to avoid detonation will improve the safety of the system. The use of more than one flow reactor, or a plurality of flow reactors, each with selected diameters that are less than the critical diameter of the explosive to be manufactured, in parallel, can increase the final output flow, without comprising safety.

Typical large bore tubular reactors used in the industrial synthesis of chemicals may have at least 3-4 mm internal diameter pipes. Whilst this allows for very large volume of reagents, the diameter is such, that should the explosive material precipitate out in the pipe and/or cause a blockage, it would allow explosive material to accumulate at a diameter greater than the critical diameter of the explosive material. This may lead to a large hazard, as the precipitated explosive could sustain detonation.

The industrial synthesis of explosive materials, may preferably have a flow synthesis flow with a pipe having an internal diameter less than 1 mm, more preferably less than 500 microns, preferably in the range of 100 to 500 microns, preferably 250 to 350 microns.

According to a further aspect of the invention there is provided an apparatus for carrying out the method of any one of the preceding claims. The apparatus comprising a plurality of flow reactors in parallel, each of said flow reactors comprising a pipe, wherein the internal diameter of the pipe is selected such that it is less than the critical diameter of the organic high explosive

According to a further aspect of the invention there is provided a method of synthesising an organic high explosive, comprising the steps of
i) providing at least one solution, comprising a nitrating agent and an explosive precursor,
ii) causing the solution to be mixed and passed through a flow reactor,
wherein the flow reactor comprises a pipe, wherein the internal diameter of the pipe is selected such that it is less than the critical diameter of the energetic material being formed, thereby preventing detonation of the energetic material in said flow reactor.

The use of flow synthesis provides a facile means of preparing RDX, HMX etc at both laboratory R&D scale of ˜100 g, and to provide the ability to add further flow reactors to readily scale up production, without the associated dangers of forming +100 Kgs of RDX explosive in a single reactor vessel. Further, it also avoids the use of hundreds of litres of highly concentrated acid in a large reactor vessel in a batch process. The use of flow synthesis allows for the continuous removal and safe stowage of final explosive product material from the flow reactor or flow reactors, to avoid the build-up of large quantities of explosive material. This may allow explosive processing buildings to process a greater mass of explosive and/or associated safety distances to be reduced, as the explosive material may be distributed to safe areas, away from the flow reactor, as it is synthesised.

### RDX Synthesis

Hexamine was added to the input flow reagent A nitric acid in any wt % up to and including a near saturated solution. The higher the concentration of hexamine in the Input flow reagent A, the more efficient the process. It is highly preferable to dissolve the hexamine in the nitric acid, as short a time as possible before flowing into the reactor, to reduce the likelihood of the nitration reaction starting.

The hexamine may be dissolved in nitric acid with a concentration in the range of from 70% to 92%, more preferably from 88% to 92%., the use of other solvents to aid dissolving the hexamine, may be added.

Preferably input flow reagent A contains only hexamine and nitric acid with a concentration in the range of less and 92%.

The input flow reagent B may comprise 99% concentration nitric acid to ensure the total nitric acid concentration in the flow reactor is at least 92% nitric acid concentration, more preferably input flow reagent B contains only 99% concentration nitric acid.

The use of nitric acid at a concentration below that at which nitration can occur, allows the hexamine starting material to be dissolved, without the nitration reaction starting. This prevents product from precipitating out before it is flowed into the flow reactor, and may prevent blockage of the flow reactor and associated mixing chambers. Further, the use of high percentage concentrations as the dissolving agent for hexamine permits the reaction in the flow reactor to be quickly brought up to the required total nitric acid concentration for nitration to occur. This avoids the issue of having a diluted concentration of nitric acid concentration in the flow reactor, therefore the input flow reagent B only needs to be a slightly higher concentration of nitric acid, to ensure that the desired range of total nitric acid concentration of greater than 92% is achieved in the flow reactor.

To assist in achieving the desirable concentration of nitric acid to start nitration of hexamine, after step ii, the input flow reagents A and B may be premixed in a mixing chamber before entering the flow reactor.

It has been found that in step iii) the total nitric acid concentration may be in the range of 90-99% in said flow reactor, more preferably in the range of 93% to 95% nitric acid concentration.

The total nitric acid concentration when input flow reagent A and input flow reagent B contain only nitric acid as the acid and the sole nitration agent, the concentration must be sufficient for nitration to occur, such as for example greater than 92% concentration.

The flow rate of input flow reagent A may be selected from any suitable flow rate with input flow reagent B, to provide a total nitric acid concertation capable of causing nitration of hexamine, such as for example in the range of greater than 92%. The actual flow rate of input flow reagent A may be μL through to millilitres to litres, depending on the capacity of the flow cell.

The flow rate of input flow reagent B may be selected from any suitable flow rate with input flow reagent A to provide a total nitric acid concertation capable of causing nitration of hexamine, such as for example in the range of greater than 92% concentration. The actual flow rate of input flow reagent A may be μL through to millilitres to litres, depending on the capacity of the flow cell. The ratio of the flow rate Input flow reagent A to Input flow reagent B (A:B) may be B>A, preferably the ratio is greater than 1:3 (A:B), more preferably in the range of (1:4) to (1:10), to ensure the nitric acid total concentration is greater than 92% in the flow reactor. The use of higher concentrations of acid in Input flow reagent B, allows the volume/flow rate of Input flow reagent B to be reduced, ie a lower ratio, which may lead to reducing the quantity of nitric acid being used. This may be caused by using other strong acids, such as for example oleum.

The temperature in the flow reactor needs to be controlled to prevent a highly exothermic reaction from occurring, preferably the temperature is caused to be less than 30° C., preferably between 20° C. to 30° C., more preferably between 22° C. to 27° C., most preferably at 24° C. The temperature is monitored by water circulators. The flow reactor may be cooled by any suitable means such as for example water circulator or electric coolers.

The reaction in step v, the output mixed flow is quenched, to stop the reaction and to cause precipitation of the RDX product. The output flow may be transferred in to a large volume of quench medium or mixed in a mixing chamber.

Preferably the output mixed flow, which comprises the RDX dissolved in the nitric acid, is mixed with the quench medium via an SOR mixer at the end of the flow reactor. The quench medium may have a pH 7 or less, and may be selected from an aqueous acidic solution or water. The quenching agent may be cooled to induce crystallisation, preferably less than 20° C., preferably in the region of 10° C. or less.

The RDX precipitate is filtered and collected and then washed in an aqueous solution, preferably the quenching solution may have a pH 7 or less, preferably water.

The nitration reagent may be selected from at least 70% concentration nitric acid and NaNO₂, or containing only 99% concentration nitric acid.

According to a further aspect of the invention there is provided a method of synthesising an organic high explosive, comprising the steps of
i) providing a first solution A
ii) providing a second solution B,
wherein the admixture of solution A and solution B are selected such that they are capable upon formation of the admixture of reacting together to provide an organic high explosive,
iii) causing the solution A and B to be mixed and passed through a flow reactor to create an admixture,
   wherein the flow reactor comprises a pipe, wherein the internal diameter of the pipe is selected such that it is less than the critical diameter of the organic high explosive, thereby preventing detonation of the formed organic high explosive in said flow reactor.

### Experimental Reagents for RDX/HMX

99% HNO3 was purchased from Honeywell in a 500 mL quantity. Cat. 84392-500ML, Lot. No. 1345S.

70% HNO3 was purchased from Fisher scientific in a 2.5 L quantity. Code: N/2300/PB17. Lot: 1716505.

Hexamine was purchased from Sigma-Aldrich in a 250 g quantity. Cat. 797979-250G, Lot. No. MKCJ7669.

Oleum was purchased from Fisher in a 500 mL quantity. Cat. S/9440/PB08, Lot. No. 1689177.

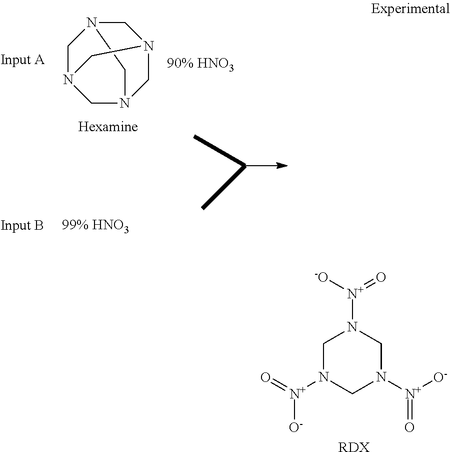

The general reaction is shown above, where the input flow reagent A comprises hexamine dissolved in nitric acid, and input flow reagent B comprises the nitrating agent, which may be higher concentration of nitric acid (than input flow reagent A), and/or a further nitrating agent, such as a metal nitrite, such as NaNO₂. The input flow reagent A and input flow reagent B are caused to react in the flow reactor to furnish the product RDX.

RDX synthesis using a flow reactor poses more challenging design issues than simply pumping solutions from well-known and quantified batch chemistry. This is mainly due to the fact that the starting material hexamine is solid, and RDX can potentially precipitate out of solution during the reaction. Precipitation of the RDX during the transition through the flow reactor can happen as the acid concentration drops and water content increases, thereby leading to potential blockages in the flow reactor, this could lead to catastrophic events, and so the nitric acid concentration in the flow chemistry.

Before starting the experiment the reactor was prepared by flushing the system with methanol followed by water. Both input systems were then filled with 70% HNO₃ which was passed through the reactor in order to fully prime the system.

### EXPERIMENT 1

Syringe A: Saturated hexamine in 90% HNO₃ (roughly 1 g in 5 mL).
Syringe B: 99% HNO₃.
Flow reactor used: 3222 Labtrix

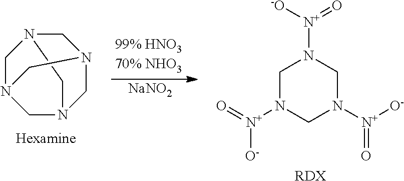

The concentration of the nitric acid in syringe A was 90% conc. The flow rate was set at 1:3 (A:B), however limited product formed. The flow rate of syringe B, 99% HNO₃ feed was increased so that the A:B flow ratio was 1:9. When the sample was collected into water the solution became opaque indicating that RDX had been produced.

### EXPERIMENT 2 ADDITION OF OLEUM

Syringe A: 0.5 g hexamine dissolved in 2.5 mL 90% HNO₃. Solution cooled during hexamine addition.
Syringe B: 0.95 mL 99% HNO₃+0.05 mL oleum.

A series of experiments were carried out aimed at monitoring the influence of oleum on RDX formation. Experiments produced opaque solutions when collected into water indicative of RDX formation. The ¹H NMR spectrum showed that RDX exists in solution prior to precipitation using water as the quenching agent.

### EXPERIMENT 3 INCREASED OLEUM ADDITION

Syringe A: 0.5 g hexamine dissolved in 2.5 mL 90% HNO₃. Solution cooled during hexamine addition.
Syringe B: 0.9 mL 99% HNO₃+0.1 mL oleum.

The increase of oleum by 100%, led to formation of RDX precipitate when collected onto ice. Part of the solution before mixing with ice was collected into d⁶-DMSO, the ¹H NMR spectrum indicated the formation of RDX.

The use of further acids such as oleum, helps to keep that acid concentration in the reactor at a high level, and may assist in dehydration of the reaction. The use of nitration species such as NaNO₂, can allow the use of lower total nitric acid concentrations.

It was found that low acidity in the reactor caused RDX to precipitate from the solution. It is essential to monitor the flow reactor paths for solidified product. Further, whilst it is desirable to increase the acidity of the nitric acid that comprises the hexamine, if the concentration is too high product starts to form, before mixing has commenced, again leading to likelihood of RDX product blocking the flow reactor. Preferably the hexamine is dissolved in the nitric acid, before use, and is not stored long term as a stock solution.

### HMX Example

TAT can be readily synthesised from hexamine via DAPT as an intermediate. The main advantage of going through this route is that an 8-membered ring is formed therefore eliminating the possibility of forming RDX as a biproduct. The TAT can be directly converted to HMX, using nitration in a flow synthesis arrangement to control the rate of production of explosive material.

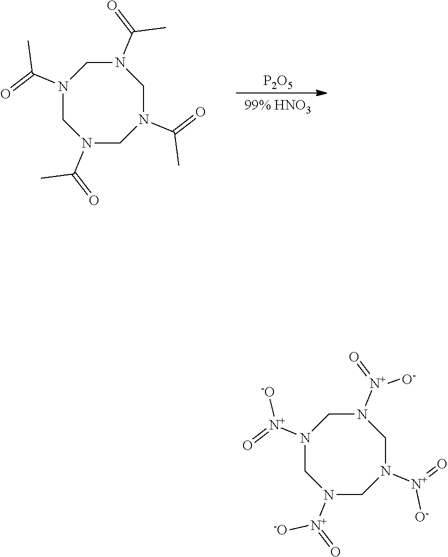

### EXPERIMENTAL 1

Line A—solution of 100 mg TAT, 1000 mg P₂O₅ and 2 mL of 99% HNO₃ were premixed in a single solution and passed through a Labtrix reactor. The preferred reaction time of 120 seconds through the flow synthesis reactor provided sufficient time for nitration to occur. The flow synthesis was performed at a temperature greater than room temperature, it was found that a temperature of 75° C. provided a temperature which allowed the reaction to proceed, but not sufficient to cause an unwanted explosive event. HMX was isolated, without any RDX contamination being present.

### EXPERIMENT 2 A SCALED UP PROTRIX REACTOR

Line A: 2.0061 g TAT and 20.0357 g of P₂O₅ were dissolved in 40 mL 99% HNO₃
Line B was used as an emergency flush and was primed with 70% HNO₃.

Line A and the Protrix were initially primed with 70% HNO₃ followed by 99% HNO₃. The reaction mixture was prepared in stages. Initially P₂O₅ is slowly dissolved into a stirred solution of 99% HNO₃. This solution was kept in an ice bath. This resulted in an opaque-yellow solution. The addition of TAT to this solution reduced the opacity of the solution however, the reaction mix remained opaque.

Line A was then primed with the reaction mixture.

| | TEMP | TIME | FLOW | | |
|---|---|---|---|---|---|
| EXP | (° C.) | (S) | A (ML) | P (BAR) | OBSERVATION |
| 0168 | 75 | 120 | 1.66 | 1.1 | Collected for 12 |
| | | | | | minutes into ice. |

The solution from the Protrix was left overnight, which resulted in the formation of crystals. These were isolated, washed with water followed by acetone and then analysed using NMR spectroscopy. The ¹H NMR spectrum of experiment 0168 shows that there are multiple species present in the sample. Some of these peaks correspond to unreacted TAT and partially nitrated TAT. These impurities are also observed in the industrial batch synthesis of HMX from TAT and can be removed by boiling the material in acetone followed by recrystallisation. In the 1H NMR spectrum, a peak at 6.02 ppm is characteristic of HMX.

1. A method of synthesizing an organic high explosive in a flow reactor including a pipe, the method comprising:
combining a solution A comprising a nitrating agent with a solution B comprising an explosive precursor reagent to produce an admixture, wherein solution A and solution B are selected such that they are capable upon formation of the admixture of reacting together to provide the organic high explosive;
determining a critical diameter of the organic high explosive;
selecting an internal diameter of the pipe such that it is less than the critical diameter of the organic high explosive, thereby preventing detonation of the formed organic high explosive in said flow reactor; and
causing the solution A and B to be mixed and passed through the flow reactor to create the admixture to provide the organic high explosive.

2. The method according to claim 1, wherein the nitrating agent comprises nitric acid and/or nitrites.

3. The method according to claim 1, wherein the organic high explosive is a nitramine.

4. The method according to claim 3, wherein the nitramine is RDX or HMX.

5. The method according to claim 1, wherein the explosive precursor reagent is selected from at least one of: cycloamine, Octahydro-1,3,5,7-tetranitro-1,3,5,7-tetrazocine (TAT), 1,3,5-triacetyl-1,3,5-triazacyclohexanes (TRAT), 1,5-Dinitroendomethylene-1,3,5,7-tetraazacyclooctane (DPT), and hexamethylenetetramine.

6. The method according to claim 1, wherein solution A and/or solution B and/or a solution C mixed to create the admixture further comprises catalysts, strong acids, de-hydrating agents, and/or acid anhydrides.

7. The method according to claim 1, wherein the flow reactor is temperature controlled.

8. The method according to claim 1, wherein after the admixture has transitioned through the flow reactor, a solution D is added to work up the reacted admixture to provide a precipitate of said organic high explosive or a salt thereof.

9. The method according to claim 8 wherein solution D may comprise cooled water.

10. The method according to claim 1, wherein the internal diameter of the pipe is less than 500 microns.

11. The method according to claim 1, wherein the organic high explosive is collected remotely from at least stored solutions A and/or B, such as to reduce the hazard of an event.

12. The method according to claim 11, wherein the collected remotely organic high explosive is behind a blast wall or within an explosive magazine.

13. An apparatus for carrying out the method of claim 1, comprising a plurality of flow reactors in parallel, each of said flow reactors comprising a pipe, wherein the internal diameter of the pipe is selected such that it is less than the critical diameter of the organic high explosive.

14. The apparatus of claim 13, wherein the internal diameter is less than 500 microns.

15. The method according to claim 2, wherein the organic high explosive is nitramine.

16. The method according to claim 15, wherein the nitramine is RDX or HMX.

17. The method according to claim 3, wherein the explosive precursor reagent is selected from: cycloamine, octahydro-1,3,5,7-tetranitro-1,3,5,7-tetrazocine (TAT), 1,3,5-triacetyl-1,3,5-triazacyclohexanes (TRAT), 1,5-dinitroendomethylene-1,3,5,7-tetraazacyclooctane (DPT), and/or hexamethylenetetramine.

18. The method according to claim 17, wherein solution A and/or solution B and/or the admixture further comprises catalysts, strong acids, de-hydrating agents, and/or acid anhydrides.

19. The method according to claim 17, wherein after the admixture has transitioned through the flow reactor, a solution D is added to work up the reacted admixture to provide a precipitate of said organic high explosive or a salt thereof.

20. The method according to claim 19, comprising collecting the organic high explosive remotely from stored solutions A and/or B and/or C, such as to reduce the hazard of an event.
